(19) United States
(12) Patent Application Publication
(10) Pub. No.: US 2007/0161560 A1
(43) Pub. Date: Jul. 12, 2007

(54) Use of vanilloid 4 receptor and antagonists or agonists thereof for treating diseases associated with pain

(75) Inventors: John Beresford Davis, Harlow (GB); Martin James Gunthorpe, Harlow (GB); Julie Egerton, Harlow (GB); Darren Smart, Harlow (GB)

Correspondence Address: GLAXOSMITHKLINE;Corporate Intellectual Property - UW2220, P.O. Box 1539, King of Prussia, PA, 19406-0939, US

(73) Assignee: SmithKline Beecham plc

(21) Appl. No.: 11/549,189

(22) Filed: Oct. 13, 2006

Related U.S. Application Data
(63) Continuation of application No. 10/415,570, filed on Sep. 12, 2003, now abandoned.

(30) Foreign Application Priority Data
Oct. 25, 2000 (GB) 0026114.9

(51) Int. Cl. A61K 48/00; A61K 38/17; C12Q 1/68; G01N 33/567

(52) U.S. Cl. 514/12; 514/44; 435/6; 435/7.2

(57) ABSTRACT

The use of VR4 polypeptides and polynucleotides in the design of protocols for the treatment of diseases of cartilage, such as hyaline-, fibro- and elastic-cartilage, or diseases of tissues where such cartilage is found including diseases or disorders affecting the larynx, auditory canal, intervertebral discs, ligaments, tendons and joint capsules, bone development including osteoporosis, diseases involving joint destruction and also pain linked to rheumatoid arthritis and osteoarthritis.

### CROSS REFERENCE TO PRIOR APPLICATIONS

This is a continuation of U.S. patent application Ser. No. 10/415,570 filed 12 Sep. 2003 which is a 371 application of PCT/GB01/04739 filed 25 Oct. 2001 which claims the benefit of GB application 0026114.9 filed 25 Oct. 2000.

### FIELD OF THE INVENTION

This invention relates to new uses for polynucleotides and polypeptides encoded by them, to their use in therapy and in identifying compounds which may be agonists, antagonists and/or inhibitors which are potentially useful in therapy.

### SUMMARY OF THE INVENTION

In one aspect, the invention relates to new uses of the vanilloid 4 receptor (hereinafter VR4) polypeptide. Such uses include the identification and development of compounds useful in the treatment of diseases of cartilage such as hyaline-, fibro- and elastic-cartilage, or diseases of the tissues where these are found. Examples of such diseases include, but are not limited to, diseases or disorders affecting the larynx, auditory canal, intervertebral discs, ligaments, tendons, joint capsules or bone development including osteoporosis. In particular the invention concerns diseases involving joint destruction and also pain linked to rheumatoid arthritis and osteoarthritis. These disease indications are referred to herein as “the diseases”. In a further aspect, the invention relates to methods for treating conditions associated with VR4 imbalance or mutation with the identified compounds. In a still further aspect, the invention relates to diagnostic assays for detecting diseases or disorders associated with inappropriate VR4 activity or levels.

### DESCRIPTION OF THE INVENTION

A cDNA encoding human VR4 has recently been described (patent application no.EP202352.1 SmithKline Beecham; Strotmann, R. et al (2000) Nature Cell Biology 2: 695-702). VR4 has a similar predicted structure to vanilloid receptor-1 (VR1), possessing an N-terminal domain containing ankyrin repeats, six transmembrane domains and a predicted pore loop between the fifth and sixth transmembrane domains. Various cDNAs have been published which may represent splice variants of the human VR4 gene, for example Delany, N. S., et al (2000) Eur. J. Neurosci. 12: suppl. 11, 134.10 p 306 who also shows expression of VR4 in pancreas, prostate, placenta and trachea. In addition species homologues comprising regions of homology to human VR4 are known, for example Suzuki, M., et al (1999) J. Biol. Chem. 275: 2756-2762.

The present invention is based on the surprising finding that VR4 is expressed at significantly higher levels in articular cartilage than in a wide range of other tissues tested. In addition isolated chondrocytes are shown to have an unusually high level of VR4 mRNA expression. The invention concerns the use of VR4 polypeptides and the polynucleotides encoding the polypeptides in the treatment of diseases involving cartilage. The polypeptides may be used directly in such treatment or may be used in screens to identify compounds useful in such treatment.

Thus in a first aspect, the present invention relates to the use of a compound selected from:

(a) a VR4 polypeptide;

(b) a compound which modulates the activity of a VR4 polypeptide;

(c) a polynucleotide encoding a VR4 polypeptide; or

(d) an antisense polynucleotide to a polynucleotide encoding a VR4 polypeptide,

for the manufacture of a medicament for treating diseases of cartilage and/or bone, or for the treatment of pain associated therewith.

Such diseases of cartilage include those involving hyaline-, fibro- and elastic-cartilage, or diseases of tissues where such cartilage is found including diseases or disorders affecting the larynx, auditory canal, intervertebral discs, ligaments, tendons and joint capsules. Diseases of bone include those of bone development including osteoporosis.

In a preferred embodiment the disease is one involving joint destruction, preferably rheumatoid arthritis or osteoarthritis.

In a further preferred embodiment the disease concerns pain associated with a disease involving joint destruction, preferably rheumatoid arthritis and osteoarthritis.

Compounds which modulate the activity of a VR4 polypeptide include compounds that activate the VR4 polypeptide and also compounds which inhibit the activity of a VR4 polypeptide.

VR4 polypeptides for use in the invention, either directly in the manufacture of a medicament or indirectly, for example when used in a screen to identify modulators of VR4 activity, include isolated polypeptides comprising an amino acid sequence which has at least 95% identity, preferably at least 97-99% identity, to that of SEQ ID NO:2. Such polypeptides include those comprising the amino acid of SEQ ID NO:2.

Further VR4 polypeptides include isolated polypeptides in which the amino acid sequence has at least 95% identity, preferably at least 97-99% identity, to the amino acid sequence of SEQ ID NO:2. Such polypeptides include the polypeptides of SEQ ID NO:2.

Still further VR4 polypeptides include isolated polypeptides encoded by a polynucleotide comprising the sequence contained in SEQ ID NO:1.

The VR4 polypeptides may be in the form of the “mature” protein or may be a part of a larger protein such as a precursor or a fusion protein. It is often advantageous to include an additional amino acid sequence which contains secretory or leader sequences, pro-sequences, sequences which aid in purification such as multiple histidine residues, or an additional sequence for stability during recombinant production.

The VR4 polypeptides can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

For preparing VR4 polypeptides by recombinant means, a polynucleotide encoding a VR4 polypeptide can be used (hereinafter a “VR4 polynucleotide”).

VR4 polynucleotides may be obtained, using standard cloning and screening techniques (Sambrook et at., Molecular Cloning: A Laboratory Manual, 2nd Ed., Cold Spring Harbor Laboratory Press, Cold Spring Harbor, N.Y. (1989) and European patent application no. EP202352.1 SmithKline Beecham), from a cDNA library derived from mRNA in cells of human osteoarthritic cartilage, heart, kidney or human brain. VR4 polynucleotides can also be obtained from natural sources such as genomic DNA libraries or can be synthesized using well known and commercially available techniques. EP202352.1 further discloses methods for the recombinant production of VR4 polypeptides, including expression vectors and hosts and details of purification methods. This invention also relates to the use of polynucleotides of the present invention as diagnostic reagents useful in the detection of diseases caused by over or underexpression of VR4 polypeptide, or expression of a mutated form of VR4, in a subject. Such diseases include diseases of cartilage, such as hyaline-, fibro- and elastic-cartilage, or diseases of tissues where such cartilage is found including diseases or disorders affecting the larynx, auditory canal, intervertebral discs, ligaments, tendons and joint capsules, bone development including osteoporosis, diseases involving joint destruction and also pain linked to rheumatoid arthritis and osteoarthritis.

Detection of a mutated form of the gene characterised by the polynucleotide of SEQ ID NO:1 which is associated with a dysfunction will provide a diagnostic tool that can add to, or define, a diagnosis of a disease, or susceptibility to a disease, which results from under-expression, over-expression or altered expression of the VR4 gene. Individuals carrying mutations in the gene may be detected at the DNA level by a variety of techniques.

Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled VR4 nucleotide sequences. Perfectly matched sequences can be distinguished from mismatched duplexes by Rnase digestion or by differences in melting temperatures. DNA sequence differences may also be detected by alterations in electrophoretic mobility of DNA fragments in gels, with or without denaturing agents, or by direct DNA sequencing (ee, e.g., Myers et al, Science (1985) 230:1242). Sequence changes at specific locations may also be revealed by nuclease protection assays, such as Rnase and S1 protection or the chemical cleavage method (see Cotton et al, Proc Natl Acad Sci USA (1985) 85: 4397-4401). In another embodiment, an array of oligonucleotides probes comprising VR4 nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability (see for example: M. Chee et al., Science, Vol 274, pp 610-613 (1996)).

The diagnostic assays offer a process for diagnosing or determining a susceptibility to the Diseases through detection of mutation in the VR4 genes by the methods described. In addition, such diseases may be diagnosed by methods comprising determining from a sample derived from a subject an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, Rnase protection, Northern blotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays.

Thus in another aspect, the present invention relates to a diagnostic kit which comprises:

(a) a polynucleotide of the present invention, preferably the nucleotide sequence of SEQ ID NO:1 or a fragment thereof;

(b) a nucleotide sequence complementary to that of (a);

(c) a polypeptide of the present invention, preferably the polypeptide of SEQ ID NO:2 or a fragment thereof; or

(d) an antibody to a polypeptide of the present invention, preferably to the polypeptide of SEQ ID NO:2.

It will be appreciated that in any such kit, (a), (b), (c) or (d) may comprise a substantial component. Such a kit will be of use in diagnosing diseases of cartilage, such as hyaline-, fibro- and elastic-cartilage, or diseases of tissues where such cartilage is found including diseases or disorders affecting the larynx, auditory canal, intervertebral discs, ligaments, tendons and joint capsules, bone development including osteoporosis, diseases involving joint destruction and also pain linked to rheumatoid arthritis and osteoarthritis.

VR4 polypeptides or their fragments or analogs thereof, or cells expressing them, can also be used as immunogens to produce antibodies immunospecific for polypeptides of the present invention. The term “immunospecific” means that the antibodies have substantially greater affinity for the polypeptides of the invention than their affinity for other related polypeptides in the prior art.

Antibodies generated against VR4 polypeptides may be obtained by administering the polypeptides or epitope-bearing fragments, analogs or cells to an animal, preferably a non-human animal, using routine protocols. For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozbor et al, Immunology Today (1983) 4:72) and the EBV-hybridoma technique (Cole et al, Monoclonal Antibodies and Cancer Therapy, 77-96, Alan R. Liss, Inc., 1985).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies.

Antibodies against polypeptides of the present invention may be employed to diagnose or treat diseases of cartilage, such as hyaline-, fibro- and elastic-cartilage, or diseases of tissues where such cartilage is found including diseases or disorders affecting the larynx, auditory canal, intervertebral discs, ligaments, tendons and joint capsules in addition to bone development, including osteoporosis, and diseases involving joint destruction and also pain linked to rheumatoid arthritis and osteoarthritis.

Another aspect of the invention relates to a method for inducing an immunological response in a mammal which comprises inoculating the mammal with a polypeptide of the present invention, adequate to produce antibody and/or T cell immune response to protect said animal from the diseases hereinbefore mentioned, amongst others. Yet another aspect of the invention relates to a method of inducing immunological response in a mammal which comprises, delivering a polypeptide of the present invention via a vector directing expression of the polynucleotide and coding for the polypeptide in vivo in order to induce such an immunological response to produce antibody to protect said animal from diseases.

A further aspect of the invention relates to an immunological/vaccine formulation (composition) which, when introduced into a mammalian host, induces an immunological response in that mammal to a polypeptide of the present invention wherein the composition comprises a polypeptide or polynucleotide of the present invention. The vaccine formulation may further comprise a suitable carrier. Since a polypeptide may be broken down in the stomach, it is preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation instonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

VR4 polypeptides can be used to devise screening methods to identify compounds which modulate the activity of said VR4 polypeptides. Such modulators include compounds which stimulate (agonists) or inhibit (antagonists) the function of the VR4 polypeptides. Accordingly, in a further aspect, the present invention provides for a method of screening compounds to identify those which stimulate or which inhibit the function of the VR4 polypeptides. In general modulators of VR4, such as agonists or antagonists, may be employed for therapeutic and prophylactic purposes for such diseases as hereinbefore mentioned. Compounds may be identified from a variety of sources, for example, cells, cell-free preparations, chemical libraries, and natural product mixtures. Such modulators so-identified may be natural or modified substrates, ligands or receptors of the VR4 polypeptides; or may be structural or functional mimetics thereof (see Coligan et al, Current Protocols in Immunology 1(2):Chapter 5 (1991)).

The screening method may simply measure the binding of a candidate compound to the VR4 polypeptides, or to cells or membranes bearing the VR4 polypeptide, or a fusion protein thereof by means of a label directly or indirectly associated with the candidate compound. Alternatively, the screening method may involve competition with a labeled competitor. Further, these screening methods may test whether the candidate compound results in a signal generated by activation or inhibition of the VR4 polypeptides, using detection systems appropriate to the cells bearing the VR4 polypeptide. Inhibitors of activation are generally assayed in the presence of a VR4 agonist, and the effect on activation by the agonist by the presence of the candidate compound is observed. Constitutively active polypeptides may be employed in screening methods for inverse agonists or inhibitors, in the absence of an agonist or inhibitor, by testing whether the candidate compound results in inhibition of activation of the VR4 polypeptide. Further, the screening methods may simply comprise the steps of mixing a candidate compound with a solution containing a VR4 polypeptide to form a mixture, measuring VR4 activity in the mixture, and comparing the VR4 activity of the mixture to a standard. Fusion proteins, such as those made from Fc portion and VR4 polypeptide, as hereinbefore described, can also be used for high-throughput screening assays to identify antagonists for the polypeptide of the present invention (see D. Bennett et al., J Mol Recognition, 8:52-58 (1995); and K. Johanson et al., J Biol Chem, 270(16):9459-9471 (1995)).

The polynucleotides, polypeptides and antibodies to the VR4 polypeptides may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agents which may inhibit or enhance the production of polypeptide (also called antagonist or agonist, respectively) from suitably manipulated cells or tissues.

Examples of potential polypeptide antagonists include antibodies or, in some cases, oligonucleotides or proteins which are closely related to the ligands, substrates or receptors of the VR4 polypeptide, e.g., a fragment of the ligands, substrates or receptors or small molecules which bind to the VR4 polypeptides of the present invention but do not elicit a response, so that the activity of the VR4 polypeptide is prevented.

Thus, in another aspect, the present invention relates to a screening kit for identifying agonists, antagonists, ligands, receptors, substrates etc. for VR4 polypeptides; or compounds which decrease or enhance the production of such VR4 polypeptides, which comprises:

(a) a VR4 polypeptide;

(b) a recombinant cell expressing a VR4 polypeptide;

(c) a cell membrane expressing a VR4 polypeptide; or

(d) antibody to a VR4 polypeptide;

which polypeptide is preferably that of SEQ ID NO:2.

It will be appreciated that in any such kit, (a), (b), (c) or (d) may comprise a substantial component.

It will be readily appreciated by the skilled artisan that a VR4 polypeptide may also be used in a method for the structure-based design of a compound that modulates the activity of the VR4 polypeptide, by:

(a) determining in the first instance the three-dimensional structure of the VR4 polypeptide;

(b) deducing the three-dimensional structure for the likely reactive or binding site(s) of a modulating compound;

(c) synthesizing candidate modulating compounds that are predicted to bind to or react with the deduced binding or reactive site; and

(d) testing whether the candidate compounds are indeed modulators.

It will be further appreciated that this will normally be an iterative process.

In a further aspect, the present invention provides methods of treating abnormal conditions such as, for instance diseases of cartilage, such as hyaline-, fibro- and elastic-cartilage, or diseases of tissues where such cartilage is found including diseases or disorders affecting the larynx, auditory canal, intervertebral discs, ligaments, tendons and joint capsules in addition to bone development including osteoporosis, diseases involving joint destruction and also pain linked to rheumatoid arthritis and osteoarthritis, related to either an excess of, or an under-expression of, VR4 polypeptide activity.

If the activity of the VR4 polypeptide is in excess, several approaches are available. One approach comprises administering to a subject in need thereof an inhibitor compound (antagonist) as hereinabove described, optionally in combination with a pharmaceutically acceptable carrier, in an amount effective to inhibit the function of the VR4 polypeptide, such as, for example, by blocking the binding of ligands, substrates, receptors, enzymes, etc., or by inhibiting a second signal, and thereby alleviating the abnormal condition. In another approach, soluble forms of the VR4 polypeptide still capable of binding the ligand, substrate, enzymes, receptors, etc. in competition with endogenous polypeptide may be administered. Typical examples of such competitors include fragments of the VR4 polypeptide.

In still another approach, expression of the gene encoding endogenous VR4 polypeptide can be inhibited using expression blocking techniques. Known such techniques involve the use of antisense sequences, either internally generated or externally administered (see, for example, O'Connor, J Neurochem (1991) 56:560 in Oligodeoxynucleotides as Antisense Inhibitors of Gene Expression, CRC Press, Boca Raton, Fla. (1988)). Such antisense polynucleotides are designed to comprise the antisense sequence of a polynucleotide encoding a VR4 polypeptide, or a fragment thereof. A VR4 encoding polynucleotide can include a DNA or an RNA, for example a mRNA.

Alternatively, oligonucleotides which form triple helices (“triplexes”) with the gene can be supplied (see, for example, Lee et al., Nucleic Acids Res (1979) 6:3073; Cooney et al., Science (1988) 241:456; Dervan et al., Science (1991) 251:1360). These oligomers can be administered per se or the relevant oligomers can be expressed in vivo. Synthetic antisense or triplex oligonucleotides may comprise modified bases or modified backbones. Examples of the latter include methylphosphonate, phosphorothioate or peptide nucleic acid backbones. Such backbones are incorporated in the antisense or triplex oligonucleotide in order to provide protection from degradation by nucleases and are well known in the art. Antisense and triplex molecules synthesised with these or other modified backbones also form part of the present invention.

In addition, expression of the human VR4 polypeptide may be prevented by using ribozymes specific to the human VR4 mRNA sequence. Ribozymes are catalytically active RNAs that can be natural or synthetic (see for example Usman, N, et al., Curr. Opin. Struct. Biol (1996) 6(4), 527-33.) Synthetic ribozymes can be designed to specifically cleave the human VR4 mRNAs at selected positions thereby preventing translation of the human VR4 mRNAs into functional polypeptide. Ribozymes may be synthesised with a natural ribose phosphate backbone and natural bases, as normally found in RNA molecules. Alternatively the ribozymes may be synthesised with non-natural backbones to provide protection from ribonuclease degradation, for example, 2′-O-methyl RNA, and may contain modified bases.

For treating abnormal conditions related to an under-expression of VR4 and its activity, several approaches are also available. One approach comprises administering to a subject a therapeutically effective amount of a compound which activates a VR4 polypeptide of the present invention, i.e., an agonist as described above, in combination with a pharmaceutically acceptable carrier, to thereby alleviate the abnormal condition. Alternatively, gene therapy may be employed to effect the endogenous production of VR4 by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For an overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996). Another approach is to administer a therapeutic amount of a VR4 polypeptide of the present invention in combination with a suitable pharmaceutical carrier.

In a further aspect, the present invention provides for pharmaceutical compositions comprising a therapeutically effective amount of a VR4 polypeptide, such as the soluble form of a VR4 polypeptide of the present invention, agonist/antagonist peptide or small molecule compound, in combination with a pharmaceutically acceptable carrier or excipient. Such carriers include, but are not limited to, saline, buffered saline, dextrose, water, glycerol, ethanol, and combinations thereof. The invention further relates to pharmaceutical packs and kits comprising one or more containers filled with one or more of the ingredients of the aforementioned compositions of the invention. VR4 polypeptides and other compounds of the present invention may be employed alone or in conjunction with other compounds, such as therapeutic compounds.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdennal administration using penetrants such as bile salts or fusidic acids or other detergents. In addition, if a VR4 polypeptide or other compounds of the present invention can be formulated in an enteric or an encapsulated formulation, oral administration may also be possible. Administration of these compounds may also be topical and/or localized, in the form of salves, pastes, gels, and the like.

The dosage range required depends on the choice of peptide or other compounds of the present invention, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a VR4 polypeptide ex vivo, and for example, by the use of a retroviral plasmid vector. The cells are then introduced into the subject.

The following definitions are provided to facilitate understanding of certain terms used frequently hereinbefore.

“Antibodies” as used herein includes polyclonal and monoclonal antibodies, chimeric, single chain, and humanized antibodies, as well as Fab fragments, including the products of an Fab or other immunoglobulin expression library.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polynucleotide” generally refers to any polyribonucleotide or polydeoxyribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, Proteins—Structure and Molecular Properties, 2nd Ed., T. E. Creighton, W. H. Freeman and Company, New York, 1993; Wold, F., Post-translational Protein Modifications: Perspectives and Prospects, pgs. 1-12 in Post-translational Covalent Modification of Proteins, B. C. Johnson, Ed., Academic Press, New York, 1983; Seifter et al, “Analysis for protein modifications and nonprotein cofactors”, Meth Enzymol (1990) 182:626-646 and Rattan et al, “Protein Synthesis: Post-translational Modifications and Aging”, Ann NY Acad Sci (1992) 663:48-62).

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol 215: 403-410 (1990). The BLAST X program is publicly available from NCBI and other sources (BLAST Manual, Altschul, S., et al, NCBI NLM NIH Bethesda, Md. 20894; Altschul, S., et al., J. Mol. Biol. 215: 403-410 (1990). The well known Smith Waterman algorithm may also be used to determine identity.

Preferred parameters for polypeptide sequence comparison include the following:

1) Algorithm: Needleman and Wunsch, J. Mol. Biol. 48: 443-453 (1970)

Comparison matrix: BLOSSUM62 from Hentikoff and Hentikoff, Proc. Natl. Acad. Sci. USA. 89:10915-10919 (1992)

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Preferred parameters for polynucleotide comparison include the following:

1) Algorithm: Needleman and Wunsch, J. Mol. Biol. 48: 443-453 (1970)

Comparison matrix: matches=+10, mismatch=0

Gap Penalty: 50

Gap Length Penalty: 3

Available as: The “gap” program from Genetics Computer Group, Madison Wis. These are the default parameters for nucleic acid comparisons.

By way of example, a polynucleotide sequence of the present invention may be identical to the reference sequence of SEQ ID NO:1, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO:1 by the numerical percent of the respective percent identity (divided by 100) and subtracting that product from said total number of nucleotides in SEQ ID NO:1, or:
nₙ≦xₙ−(xₙ·y),
wherein nₙ is the number of nucleotide alterations, xₙ is the total number of nucleotides in SEQ ID NO:1, and y is, for instance, 0.70 for 70%, 0.80 for 80%, 0.85 for 85%, 0.90 for 90%, 0.95 for 95%, etc., and wherein any non-integer product of xₙ and y is rounded down to the nearest integer prior to subtracting it from xₙ. Alterations of a polynucleotide sequence encoding the polypeptide of SEQ ID NO:2 may create nonsense, missense or frameshift mutations in this coding sequence and thereby alter the polypeptide encoded by the polynucleotide following such alterations.

Similarly, a polypeptide sequence of the present invention may be identical to the reference sequence of SEQ ID NO:2, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:2 by the numerical percent of the respective percent identity (divided by 100) and then subtracting that product from said total number of amino acids in SEQ ID NO:2, or:
nₐ≦xₐ−(xₐ·y),
wherein nₐ is the number of amino acid alterations, xₐ is the total number of amino acids in SEQ ID NO:2, and y is, for instance 0.70 for 70%, 0.80 for 80%, 0.85 for 85% etc., and wherein any non-integer product of xₐ and y is rounded down to the nearest integer prior to subtracting it from xₐ.

“Homolog” is a generic term used in the art to indicate a polynucleotide or polypeptide sequence possessing a high degree of sequence relatedness to a subject sequence. Such relatedness may be quantified by determining the degree of identity and/or similarity between the sequences being compared as hereinbefore described. Falling within this generic term are the terms “ortholog”, meaning a polynucleotide or polypeptide that is the functional equivalent of a polynucleotide or polypeptide in another species, and “paralog” meaning a functionally similar sequence when considered within the same species.

“Fusion protein” refers to a protein encoded by two, often unrelated, fused genes or fragments thereof. In one example, EP-A-0 464 discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

### EXAMPLES

### Example 1—VR4 is Expressed in Cartilage and Chondrocytes

Tissue and cell expression of human VR4 was studied using TaqMan (Perkin Elmer) quantitative RT-PCR (Gibson et al. (1996) Genome Res. 1996 October; 6(10):995-1001) according to the manufacturers instructions. TaqMan reactions were conducted using probes for human GAPDH, cyclophilin and human VR4. The human VR4 probe consisted of:

5′-ATGAGGACCAGACCAACTGCA (SEQ ID NO:3) and

5′-GGAGGAAGGTGCTGAAGGTCTC (SEQ ID NO:4) flanking primers and a

5′-CACTTACCCCTCGTGCCGTGACAG (SEQ ID NO:6) fluorogenic probe.

Data were analysed using the Power Macintosh software accompanying the ABI Prism™ 7700.

Result: The data from a screen of body tissues, shown in Table 1, shows that human VR4 is most prominently expressed in cartilage. A screen of primary and clonal cell cultures shows significant expression only in chondrocytes.

TABLE 1
Relative mRNA expression in human tissues and cell-lines. The figures indicate a
quantitative score from 1 (low expression) to 5 (very high expression).

| | A | B | C | D | E | F | G | H | I | J | K | L | M | N | O | P | Q | R | S | T |
|---|---|---|---|---|---|---|---|---|---|---|---|---|---|---|---|---|---|---|---|---|
| T | 0 | 1 | 0 | 1 | 1 | 1 | 2 | 0 | 0 | 0 | 1 | 0 | 0 | 1 | 1 | 1 | 2 | 5 | 1 | 0 |
| C | 0 | 0 | 0 | 0 | 0 | 0 | 0 | 0 | 0 | 0 | 0 | 0 | 0 | 0 | 0 | 0 | 0 | 5 | 0 | 0 |

T relates to the category of different body tissues as follows:
A CNS, B pituitary, C heart, D lung, E liver, F foetal liver, G kidney, H skeletal muscle, I stomach, J intestine, K spleen, L lymphocytes, M macrophages, N adipose, O pancreas, P prostate, Q placenta, R cartilage, S bone, T bone marrow.
C relates to the category of different cell lines as follows:
A aortic smooth muscle cells, B bladder smooth muscle cells, C C20A4, D MG63, E SAOS2, F lymphocyte, G macrophage, H platelets, I neutrophil, J CHANG, K HepG2, L IMR32, M SK-N-MC, N SK-N-SH, O NT-2, P1321N1, Q C13, R primary human chondrocytes, S Hs-683, T HEK293.

The results show high-level expression in cartilage tissue (T, R) and primary human chondrocytes (C,R).

### Example 2—VR4 is Activated by 4α-phorbol-12,13 didecanoate

The VR4 cDNA was inserted into the expression vector pcDNA3.1 V5-His (Invitrogen). Wildtype HEK293 cells, or HEK293 cells transfected with the human VR4:pcDNA3.1 V5-His construct, or mock transfected cells, or bovine chondrocytes, were seeded into 96-well microtitre plates at 25,000 cells/well and cultured overnight. The cells were then incubated with 4 microM Fluo-3 for 2 hrs at room temperature in the dark. Dye loaded cells were washed 4× with Tyrodes buffer: (NaCl, 145 mM; KCl, 2.5 mM; Hepes, 10 mM; Glucose, 10 mM; MgC12, 1.2 mM; CaCl2, 1.5 mM), which also contained 0.2% BSA but not probenecid. Agonists and antagonists were also prepared in Tyrodes buffer. Cells were preincubated for 30 mins with antagonist or buffer. Agonist addition and measurement of cytoplasmic calcium concentration was performed in the FLIPR (Smart et al., (2000) Br. J. Pharmacol. 129, 227-230).

Results:

Both phorbol 12-myristate 13-acetate (PMA) and 4α-phorbol-12,13-didecanoate (4αPDD) increased intracellular calcium in HEK293-VR4 cells (Table 1) but were without effect in wild type HEK293 cells or in cells transfected with empty vector. PMA also activated VR1, but was only a partial agonist (Emax 0.46) compared to capsaicin and RTX. 4αPDD was inactive at VR1 (Table 1).

TABLE 1

| | pEC50 | | | |
|---|---|---|---|---|
| | wild type | empty vector | hVR1 | hVR4 |
| RTX | IA | IA | 8.93 ± 0.20 | IA |
| capsaicin | IA | IA | 7.48 ± 0.12 | IA |
| PMA | IA | IA | 7.86 ± 0.06 | 6.64 ± 0.06 |
| 4αPDD | IA | IA | IA | 5.73 ± 0.06 |

Data are mean ± s.e. mean, where n = 3-5.
IA = inactive
hVR1 is human VR1
hVR4 is human VR4

In conclusion, 4αPDD acts as a VR4 selective agonist.

Bovine articular chondrocytes responded to 4α-PDD with a similar dose dependency as the transfected HEK293 cells. The response to 4α-PDD had a similar kinetic profile and concentration dependency to that seen for the recombinant VR4 expressed in HEK293 cells. The response was dependent upon extracellular calcium ions and was blocked by the channel blocker ruthenium red. These data suggest that the response to 4α-PDD was due to the VR4 endogenously expressed by chondrocytes.

SEQUENCE INFORMATION

SEQ ID NO:1

ATGGCGGATTCCAGCGAAGGCCCCCGCGCGGGGCCCGGGGAGGTGGCTGA
GCTCCCCGGGGATGAGAGTGGCACCCCAGGTGGGGAGGCTTTTCCTCTCT
CCTCCCTGGCCAATCTGTTTGAGGGGGAGGATGGCTCCCTTTCGCCCTCA
CCGGCTGATGCCAGTCGCCCTGCTGGCCCAGGCGATGGGCGACCAAATCT
GCGCATGAAGTTCCAGGGCGCCTTCCGCAAGGGGGTGCCCAACCCCATCG
ATCTGCTGGAGTCCACCCTATATGAGTCCTCGGTGGTGCCTGGGCCCAAG
AAAGCACCCATGGACTCACTGTTTGACTACGGCACCTATCGTCACCACTC
CAGTGACAACAAGAGGTGGAGGAAGAAGATCATAGAGAAGCAGCCGCAGA
GCCCCAAAGCCCCTGCCCCTCAGCCGCCCCCCATCCTCAAAGTCTTCAAC
CGGCCTATCCTCTTTGACATCGTGTCCCGGGGCTCCACTGCTGACCTGGA
CGGGCTGCTCCCATTCTTGCTGACCCACAAGAAACGCCTAACTGATGAGG
AGTTTCGAGAGCCATCTACGGGGAAGACCTGCCTGCCCAAGGCCTTGCTG
AACCTGAGCAATGGCCGCAACGACACCATCCCTGTGCTGCTGGACATCGC
GGAGCGCACCGGCAACATGCGGGAGTTCATTAACTCGCCCTTCCGTGACA
TCTACTATCGAGGTCAGACAGCCCTGCACATCGCCATTGAGCGTCGCTGC
AAACACTACGTGGAACTTCTCGTGGCCCAGGGAGCTGATGTCCACGCCCA
GGCCCGTGGGCGCTTCTTCCAGCCCAAGGATGAGGGGGGCTACTTCTACT
TTGGGGAGCTGCCCCTGTCGCTGGCTGCCTGCACCAACCAGCCCCACATT
GTCAACTACCTGACGGAGAACCCCCACAAGAAGGCGGACATGCGGCGCCA
GGACTCGCGAGGCAACACAGTGCTGCATGCGCTGGTGGCCATTGCTGACA
ACACCCGTGAGAACACCAAGTTTGTTACCAAGATGTACGACCTGCTGCTG
CTCAAGTGTGCCCGCCTCTTCCCCGACAGCAACCTGGAGGCCGTGCTCAA
CAACGACGGCCTCTCGCCCCTCATGATGGCTGCCAAGACGGGCAAGATTG
GGATCTTTCAGCACATCATCCGGCGGGAGGTGACGGATGAGGACACACGG
CACCTGTCCCGCAAGTTCAAGGACTGGGCCTATGGGCCAGTGTATTCCTC
GCTTTATGACCTCTCCTCCCTGGACACGTGTGGGGAAGAGGCCTCCGTGC
TGGAGATCCTGGTGTACAACAGCAAGATTGAGAACCGCCACGAGATGCTG
GCTGTGGAGCCCATCAATGAACTGCTGCGGGACAAGTGGCGCAAGTTCGG
GGCCGTCTCCTTCTACATCAACGTGGTCTCCTACCTGTGTGCCATGGTCA
TCTTCACTCTCACCGCCTACTACCAGCCGCTGGAGGGCACACCGCCGTAC
CCTTACCGCACCACGGTGGACTACCTGCGGCTGGCTGGCGAGGTCATTAC
GCTCTTCACTGGGGTCCTGTTCTTCTTCACCAACATCAAAGACTTGTTCA
TGAAGAAATGCCCTGGAGTGAATTCTCTCTTCATTGATGGCTCCTTCCAG
CTGCTCTACTTCATCTACTCTGTCCTGGTGATCGTCTCAGCAGCCCTCTA
CCTGGCAGGGATCGAGGCCTACCTGGCCGTGATGGTCTTTGCCCTGGTCC
TGGGCTGGATGAATGCCCTTTACTTCACCCGTGGGCTGAAGCTGACGGGG
ACCTATAGCATCATGATCCAGAAGATTCTCTTCAAGGACCTTTTCCGATT
CCTGCTCGTCTACTTGCTCTTCATGATCGGCTACGCTTCAGCCCTGGTCT
CCCTCCTGAACCCGTGTGCCAACATGAAGGTGTGCAATGAGGACCAGACC
AACTGCACAGTGCCCACTTACCCCTCGTGCCGTGACAGCGAGACCTTCAG
CACCTTCCTCCTGGACCTGTTTAAGCTGACCATTGGCATGGGCGACCTGG
AGATGCTGAGCAGCACCAAGTACCCCGTGGTCTTCATCATCCTGCTGGTG
ACCTACATCATCCTCACCTTTGTGCTGCTCCTCAACATGCTCATTGCCCT
CATGGGCGAGACAGTGGGCCAGGTCTCCAAGGAGAGCAAGCACATCTGGA
AGCTGCAGTGGGCCACCACCATCCTGGACATTGAGCGCTCCTTCCCCGTA
TTCCTGAGGAAGGCCTTCCGCTCTGGGGAGATGGTCACCGTGGGCAAGAG
CTCGGACGGCACTCCTGACCGCAGGTGGTGCTTCAGGGTGGATGAGGTGA
ACTGGTCTCACTGGAACCAGAACTTGGGCATCATCAACGAGGACCCGGGC
AAGAATGAGACCTACCAGTATTATGGCTTCTCGCATACCGTGGGCCGCCT
CCGCAGGGATCGCTGGTCCTCGGTGGTACCCCGCGTGGTGGAACTGAACA
AGAACTCGAACCCGGACGAGGTGGTGGTGCCTCTGGACAGCATGGGGAAC
CCCCGCTGCGATGGCCACCAGCAGGGTTACCCCCGCAAGTGGAGGACTGA
TGACGCCCCGCTCTAG

SEQ ID NO:2

MADSSEGPRAGPGEVAELPGDESGTPGGEAFPLSSLANLFEGEDGSLSPS
PADASRPAGPGDGRPNLRMKFQGAFRKGVPNPIDLLESTLYESSVVPGPK
KAPMDSLFDYGTYRHHSSDNKRWRKKIIEKQPQSPKAPAPQPPPILKVFN
RPILFDIVSRGSTADLDGLLPFLLTHKKRLTDEEFREPSTGKTCLPKALL
NLSNGRNDTIPVLLDIAERTGNMREFINSPFRDIYYRGQTALHIAIERRC
KHYVELLVAQGADVHAQARGRFFQPKDEGGYFYFGELPLSLAACTNQPHI
VNYLTENPHKKADMRRQDSRGNTVLHALVAIADNTRENTKFVTKMYDLLL
LKCARLFPDSNLEAVLNNDGLSPLMMAAKTGKIGIFQHIIRREVTDEDTR
HLSRKFKDWAYGPVYSSLYDLSSLDTCGEEASVLEILVYNSKIENRHEML
AVEPINELLRDKWRKFGAVSFYINVVSYLCAMVIFTLTAYYQPLEGTPPY
PYRTTVDYLRLAGEVITLFTGVLFFFTNIKDLFMKKCPGVNSLFIDGSFQ
LLYFIYSVLVIVSAALYLAGIEAYLAVMVFALVLGWMNALYFTRGLKLTG
TYSIMIQKILFKDLFRFLLVYLLFMIGYASALVSLLNPCANMKVCNEDQT
NCTVPTYPSCRDSETFSTFLLDLFKLTIGMGDLEMLSSTKYPVVFIILLV
TYIILTFVLLLNMLIALMGETVGQVSKESKHIWKLQWATTILDIERSFPV
FLRKAFRSGEMVTVGKSSDGTPDRRWCFRVDEVNWSHWNQNLGIINEDPG
KNETYQYYGFSHTVGRLRRDRWSSVVPRVVELNKNSNPDEVVVPLDSMGN
PRCDGHQQGYPRKWRTDDAPL

1. A method of treating a disease of cartilage and/or bone, or for the treatment of pain associated therewith comprising administering to a mammal in need thereof:
(a) a VR4 polypeptide;
(b) a compound which modulates the activity of a VR4 polypeptide;
(c) a polynucleotide encoding a VR4 polypeptide; or
(d) an antisense polynucleotide to a polynucleotide encoding a VR4 polypeptide,

2. The method according to claim 1 wherein the disease is one affecting the larynx, auditory canal, intervertebral discs, ligaments, tendons and joint capsules; or a disease associated with bone development including osteoporosis; or diseases involving joint destruction.

3. The method according to claim 2 wherein the diseases involving joint destruction is rheumatoid arthritis or osteoarthritis.

4. The method according to claim 1 wherein the pain is associated with rheumatoid arthritis or osteoarthritis.

5. The method according to claim 1 wherein the compound which modulates the activity of a VR4 polypeptide is an agonist.

6. The method according to claim 1 wherein the compound which modulates the activity of a VR4 polypeptide is an antagonist.

7. The method according to claim 1 wherein the compound is a VR4 polypeptide which comprises a polypeptide having at least 95% identity to the VR4 polypeptide of SEQ ID NO:2.

8. The method according to claim 7 wherein the compound is the VR4 polypeptide of SEQ ID NO:2.

9. The method according to claim 1 wherein the compound comprises a polynucleotide encoding a polypeptide having at least 95% identity with the amino acid sequence of SEQ ID NO:2.

10. The method according to claim 9 wherein the polynucleotide comprises a polynucleotide having at least 95% identity with the polynucleotide of SEQ ID NO:1.

11. The method according to claim 9 wherein the polynucleotide has the polynucleotide sequence of SEQ ID NO:1.

12. A method for screening to identify compounds that stimulate or inhibit the function or level of the polypeptide as defined in claim 7 comprising a method selected from the group consisting of:
(a) measuring or, detecting, quantitatively or qualitatively, the binding of a candidate compound to the polypeptide or a fusion protein thereof by means of a label directly or indirectly associated with the candidate compound;
(b) measuring the competition of binding of a candidate compound to the polypeptide or a fusion protein thereof in the presence of a labeled competitor;
(c) testing whether the candidate compound results in a signal generated by activation or inhibition of the polypeptide, using detection systems appropriate to the polypeptide; or
(d) mixing a candidate compound with a solution containing a polypeptide of claim 2 or 4, to form a mixture, measuring activity of the polypeptide in the mixture, and comparing the activity of the mixture to a control mixture which contains no candidate compound.
